(19) United States
(12) Patent Application Publication
(10) Pub. No.: US 2005/0049678 A1
(43) Pub. Date: Mar. 3, 2005

(54) Stent

(76) Inventors: Graeme Cocks, New South Wales (AU); Geoffrey H. White, New South Wales (AU)

Correspondence Address: Raymond Sun, 12420 Woodhall Way, Tustin, CA, 92782, US

(21) Appl. No.: 10/488,901

(22) PCT Filed: Sep. 4, 2002
(86) PCT No.: PCT/AU02/01225

(30) Foreign Application Priority Data
Sep. 4, 2001 (AU) PR 7488

(51) Int. Cl. A61F 2/06

(52) U.S. Cl. 623/1.15

(57) ABSTRACT

A stent (10) which may be used in the treatment of stenosis and particularly ostial stenosis. The stent (10) includes a flange member (15) which engages the surrounding wall of an ostium and anchors the stent (10) in its target vessel. A delivery system for a stent (10) is also disclosed, the delivery system including a membrane (109) or a compression member to hold an intraluminal stent (101) in a first radially compressed state until said stent (101) is delivered to a target site.

### FIELD OF THE INVENTION

The present invention relates to a stent for use in occlusive disease and particularly occlusive disease in an area of vessel which branches into a second vessel.

### BACKGROUND OF THE INVENTION

Occlusive diseases affecting the vasculature or other vessels are common. Such diseases include atherosclerosis which is characterised by a build up of plaque from cholesterol residues. The plaque build up subsequently thickens and hardens the vessel wall to create a stenosis. The resultant narrowing of the vessel has adverse effects on blood flow through the vessel.

Stenotic plaques may occur at any location along a vessel wall and in some cases may present at a junction between two vessels. This is commonly referred to as ostial stenosis or a narrowing of the opening of a vessel. In such cases, the plaque or area of stenosis may severely compromise the flow of blood to the “downstream” vessel.

Many vessels branch from a main vessel at approximately 90°. An example is the branching of the right and left renal arteries from the abdominal aorta. Ostial stenosis may occur at the junction between the aorta and the renal artery and may predispose the patient to atheroembolisation of the visceral and peripheral vascular beds in addition to impairing blood flow to the kidney.

Current medical practices employ both invasive and non-invasive procedures to address stenosis including ostial stenosis. While the disease may be medically treated, in severe cases surgical intervention may be required. The latter includes both balloon angioplasty to break up the stenotic plaque and the delivery of an intraluminal stent to bridge the stenotic lesion.

While both procedures are commonly used, the incidence of re-stenosis in patients treated by balloon angioplasty Is unacceptably high at an estimated 40% of cases. Bridging of the stenotic lesion with a stent significantly reduces the incidence of re-stenosis.

Conventional stents may be inserted percutaneously through a distal and connecting vessel to that in which the stent is to be used. For example, the device may be inserted through the femoral artery in a catheter, where the device is intended to be used in the treatment of a stenotic lesion. Upon release of the device from the catheter it may expand to a desirable size, and may extend above and below the lesion thereby bridging that lesion.

The delivery of a stent to an ostial stenosis is a difficult procedure. In the case of stenosis at the opening or ostium of the renal arteries, in order to bridge the stenosis, the stent is required to project around 1-2 mm into the aorta. As the majority of stents are a simple tubular mesh structure, they may shift from the desired position during the procedure. In such an event, the stent will either end up positioned too far into the renal artery or projecting too far into the aorta. Similar difficulties are experienced with the placement of a stent in other regions of ostial stenosis in visceral vessels such as the mesenteric arteries and iliac arteries.

The present invention aims to provide a stent which overcomes the abovementioned problems of the prior art

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### SUMMARY OF THE INVENTION

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The present invention consists in an intraluminal stent comprising a tubular body extending from a proximal end to a distal end, said tubular body being capable of expanding or being expanded from a radially compressed state to a radially expanded state wherein, when at least in the radially expanded state, the tubular body includes at least a first flange member positioned at or adjacent to the proximal end of the tubular body and extending outwardly from said tubular body.

In one embodiment, the first flange member extends outwardly and away from the proximal end of the tubular body of the stent to give the tubular body a trumpet-like appearance.

The first flange member may be integral with the proximal end of the tubular body and may extend to an outer rim. The outer rim may form a lipped portion which may curve back in a general direction towards the distal end of the tubular body. Alternatively, the first flange member may be formed as a separate member to the remainder of the tubular body wherein the first flange member is subsequently connected to the tubular body

Preferably, the lipped portion is integral with the first flange member such that it forms a continuous curved structure extending initially outwardly and away from the proximal end of the tubular body before curving back in a general direction towards the distal end of the tubular body.

The first flange member is typically made from the same material as the remainder of the tubular body. Alternatively, the first flange member may be made from a different material from the material of the remainder of the stent.

In another embodiment, the intraluminal stent includes a second flange member positioned at or adjacent the distal end of the tubular body.

The second flange member preferably extends outwardly and away from the distal end of the tubular body of the stent.

The second flange member may be integral with the distal end of the tubular body and may extend to an outer rim. The outer rim may form a lipped portion which may curve back in a general direction towards the proximal end of the tubular body.

Preferably, the lipped portion is integral with the second flange member such that it forms a continuous curved structure extending initially outwardly and away from the distal end of the tubular body before curving back in a general direction towards the proximal end of the tubular body.

The second flange member is typically made from the same material as the remainder of the tubular body. Alternatively, the second flange member may be made from a different material than the material of the remainder of the stent. Additionally, the second flange member may be formed as a separate member to the remainder of the tubular body and subsequently connected to the tubular body.

Preferably, the first and second flange members are made during the manufacture of the intraluminal stent of the invention. The elected method for achieving such flanged members will primarily depend on the material selected to comprise the stent and the flange member although it is envisaged that both the tubular body of the stent and the flange member would be formed by laser cutting/etching of a suitable material such as stainless steel or Nitinol™. In this regard, the tubular body of the present invention may be made by providing a cylinder of the material to be used and laser cutting said material in the cylindrical form. The laser cutting typically results in the formation of a series of cells along the length of the circumference of the cylinder. The cells may be of the same size along the length of the cylinder or may vary in size along the length. This will be discussed in greater detail below.

In the embodiment wherein the tubular body is made from a shape memory material such as Nitinol™, the cylinder of material is laser cut to form the series of cells. The cylinder is then taken to the desired temperature to allow the material to achieve a “memory” of a certain expanded configuration. At this temperature, a template is used to push the area of the cylinder which is to become the at least first flange member outwardly relative to the remainder of the cylindrical tubular body. The material of the cylinder is then cooled from this temperature such that the material resumes a compressed substantially cylindrical shape without the flanged end. When the final stent made from this cylinder is inserted into a vessel of a patient, it is preferable that when the material is exposed to the body temperature of the patient it takes on its “memorised” shape, that is, a tubular body having at least a first flange member positioned at or adjacent to a proximal end of the tubular body.

In an embodiment wherein the material of the stent Is not a shape memory material, it is envisaged that a pre-formed tubular body having at least a first flange member positioned at least adjacent the proximal end of the tubular body is formed. The pre-formed tubular body may then be laser cut into a desired series of cells.

In a preferred embodiment, the cells of the tubular body may be of varying size and configuration along the length of the tubular body. For example, it is envisaged that the at least first flange member is made from a series of cells which are larger and/or more elongate than the remainder of the cells of the tubular body. Furthermore, it is preferred that the cells of the flange member in addition to being more elongate, are formed at an angle to the remainder of the cells of the tubular body.

The tubular body may further include a transition region adjacent the at least first flange member. The transition region is preferably made up of a series of cells which have a small pore size relative to the cells of the at least first flange member and the remainder of the tubular body. In this embodiment, the transition region provides an area of relatively high expansile strength. In cases where the stent is used in ostial stenosis, which will be discussed further below, the transition region has the advantage of “pinning back” an area of stenosis around the ostium of a vessel.

The at least first flange member may comprise two cells located on opposing walls of the tubular body. In this embodiment the two cells form strut members which have the effect of anchoring the stent in a vessel, particularly when the stent is used to treat ostial stenosis as further discussed below. It should be noted, however, that the present invention encompasses all possible cell patterns in the wall of the tubular body and including the at least first flange member.

While the tubular body of the stent may be formed of a thin biocompatible material such as Nitinol™ or stainless steel, other alloys such as tantalum or Elgiloy are also envisaged. The tubular body may be bare or may be coated with a material having an elastic property such that the coating material is capable of covering the tubular body in both the radially compressed state and the radially expanded state.

In a preferred embodiment of the invention, the tubular body may be formed from other suitable biocompatible materials, selected, for best results, on the basis of the material's capacity to withstand the compressive forces of the stenotic lesion and maintain patency of the vessel throughout the life of the stent.

Preferably, the intraluminal stent of the present invention is used in the treatment of ostial stenosis although it is equally envisaged that it could be used in any other form of stenosis. In ostial stenosis, the plaque or stenotic region is formed at the junction between a pre-branching vessel and a post-branching vessel. The stenotic plaque surrounds the ostium of the post-branching vessel which has the effect of narrowing the ostium of the post-branching vessel.

While the stent of the present invention may be used to treat ostial stenosis of the visceral arteries such as the renal and mesenteric arteries, the iliac artery, and the sub-clavian artery, it may also be used to treat stenotic lesions in the peripheral vasculature and the coronary circulation. However, the application of the invention for use in the treatment of stenotic disease is not to be understood as limited to the vascular system only. The stent may be used to treat stenotic lesions in other vessels including, for example, those comprising the hepato-biliary and genito-urinary tracts

In the treatment of ostial stenosis, the first flange member is preferably positioned within the pre-branching vessel. In this embodiment, the first flange member typically engages at least a portion of the wall of the pre-branching vessel which surrounds the ostium of the post-branching vessel. The remainder of the tubular body of the stent can extend into the post-branching vessel. Accordingly, the at least first flange member has the effect of anchoring the stent within the post-branching vessel thereby preventing longitudinal movement of the stent into the post-branching vessel. If such movement occurs, the stent moves away from the stenotic lesion and thereby does not have the desired function of bridging the stenotic lesion.

In the embodiment of the invention which includes a second flange member positioned adjacent the distal end of the tubular body, when the stent is used in the treatment of ostial stenosis, the second flange member is positioned within the post-branching vessel and preferably engages the wall of said post-branching vessel. This further secures the intraluminal stent within the post-branching vessel thereby preventing longitudinal movement of the stent in the vessel.

The tubular body of the stent may further include at least one engagement member. The at least one engagement member may be connected to or integral with a wall of the tubular body at a position located intermediate the proximal end and the distal end of the tubular body.

Preferably, the tubular body includes more than one engagement member which may comprise a number of spurs or other such members which extend outwardly from the tubular body of the stent. When the stent is in use, the spurs extend towards and engage with the wall of the vessel in which the stent is positioned. This has the function of further securing the stent within the vessel.

In a further embodiment, rather than a spur, the at least one engagement member may comprise a ridge or like area or a series of ridges of increased cross sectional diameter than those portions of the tubular body immediately proximal and distal each ridge. When the stent is in use, the ridge or like area or series of ridges extend(s) towards and engage(s) with the vessel wall thereby assisting in the securing of the stent in the vessel wall.

The at least one engagement member may be made from the same material as the remainder of the tubular body or may be made from a different material. It is envisaged that the at least one engagement member may be made from a shape memory material such as Nitinol™.

The connection between the at least one engagement member and the tubular body of the stent may be such that allows the at least one engagement member to occupy a first angular relationship with an adjacent part of the tubular body when the tubular body is in its compressed state wherein the at least one engagement member occupies a second and different angular relationship with the tubular body when the tubular body is in its expanded state.

The at least one engagement member is preferably created during the manufacture of the stent of the invention. Where the tubular body and the at least one engagement member are made from a shape memory material such as Nitinol™, a cylinder of Nitinol™ is taken to a desired temperature to allow the material to achieve a “memory” of a certain configuration. If the at least one engagement member is a ridge as described above, when the cylinder is taken to this temperature, a template is used to push the area of the cylinder which is to become the at least one engagement member outwardly from the remainder of the cylindrical tubular body. The material of the cylinder is then cooled from this temperature such that the material resumes its compressed and substantially cylindrical shape without the ridge. When the final stent made from this cylinder is inserted into a vessel of a patient, it is preferable that when the material is exposed to the body temperature of the patient it takes on its “memorised” shape, that is, a tubular body having at least one engagement member extending outwardly therefrom.

Alternatively, wherein the tubular body is made up of a series of cells, the at least one engagement member may comprise a number of cells linked to one another wherein said cells extend around the circumference of the tubular body. Each cell preferably has an area of weakness which upon expansion of the tubular body from the compressed state to the expanded state buckles thereby forming a rib or a series of ribs for engagement with the vessel. This embodiment may be useful wherein the tubular body is made from a material such as stainless steel.

In a further embodiment, the at least one engagement member may be made up of a series of connector members which connect the cells on either side of the at least one engagement member. In this regard, the connector members may be relatively straight members and may connect every second cell on either side of the at least one engagement member. In this embodiment, when the tubular body moves from the radially compressed state to the radially expanded state, the free ends of every second cell which are not connected by the connector members turn outwardly away from the tubular body and engage with the vessel wall.

The tubular body may be coated with materials to promote adhesion of cells or cell growth to assist in securing the device tubular body in place in the post-branching vessel It is further envisaged that the tubular body may be coated with any of a number of pharmaceutical agents.

In a preferred embodiment, during use of the intraluminal stent of the present invention, the tubular body is initially in the radially compressed state to enable delivery of the stent through an introducer catheter. Upon deployment of the stent into a selected vessel, the tubular body may be caused to expand, or may be allowed to self-expand into the expanded state.

There are at least three preferred mechanisms whereby the tubular body may change from the radially compressed state to the radially expanded state. For instance, the tubular body may be expanded by the force of an inflating balloon within the tubular body or by some other mechanically applied force.

Alternatively, the tubular body may be made from a shape memory material as mentioned above wherein the patient's body temperature causes the temperature of the tubular body to move towards the same temperature, thereby enabling the tubular body to self-expand and take on its “memorised” shape.

In a further embodiment, the tubular body may self expand following deployment of the tubular body from an introducer catheter used to introduce the stent invention into the body of a patient. This particular embodiment relies upon spring expansion of the material of the tubular body following release of the compressive force of the introducer catheter.

The first and/or the second flange member may expand by a different mechanism to the mechanism of expansion of the remainder of the tubular body. For example the flange member(s) may be made from a shape memory material such as Nitinol™ whereas the remainder of the tubular body may be made from a spring expandable material such as stainless steel. In this case, upon deployment of the stent within a vessel, the flange members would take on their “memorised” shape and the remainder of the body would spring into shape having been compressed in the delivery catheter. Various combination of the above mechanisms are envisaged.

In a second aspect, the invention relates to a method of positioning an intraluminal stent according to the first aspect of the invention in a vessel of a patient, the method comprising the steps of:

(i) introducing a catheter or other delivery device into a vein, artery or other vessel in the body of a patient when the tubular body of the intraluminal stent is in the radially compressed state;

(ii) causing the intraluminal stent to be carried through the catheter or other delivery device to a target. site of stenosis at a bifurcation between a first pre-branching vessel and a second post-branching vessel;

(iii) causing or allowing the tubular body of the intraluminal stent to expand such that the at least first flange member is positioned at least partially within the pre-branching vessel and the remainder of the tubular body of the stent extends into the post-branching vessel; and

(iv) withdrawing the catheter or other delivery device along with any other apparatus used to introduce the intraluminal device into the vessel from the body of the patient.

The length and radially expanded diameter of the tubular body may be determined by the individual circumstances of the application to which the intraluminal device is to be put. Typically, the bifurcating vessel is assessed by X-ray or other similar examination and a suitably dimensioned device selected for that application.

The Intraluminal stent may have radio-opaque markers incorporated into the tubular body to enable a surgeon to view the position of the stent within the vessels.

In a third aspect, the present invention provides a delivery system for the delivery of the intraluminal stent of the first aspect to a target vessel, said delivery system comprising an introducer catheter having an elongate tubular body to allow the passage therethrough of a placement catheter, said placement catheter having an elongate body which extends from a proximal end to a distal end and which carries the stent of the first aspect of the invention at a position intermediate said proximal end and said distal end, the delivery system further comprising a membrane which engages a portion of the tubular body of the intraluminal stent not comprising the at least first flange member and wherein said membrane acts to maintain said portion of the tubular body in its radially compressed state.

In a fourth aspect, the present invention provides a method of delivering the intraluminal stent of the first aspect using the delivery system of the third aspect, said method comprising the steps of:

(i) introducing the introducer catheter into a vein, artery or other vessel in the body of a patient wherein the tubular body of the intraluminal stent is in the radially compressed state;

(ii) causing the intraluminal stent, the placement catheter and the membrane to be carried through the introducer catheter to a target site of stenosis at a bifurcation between a first pre-branching vessel and a second post-branching vessel;

(iii) introducing the distal end of the placement catheter into the post-branching vessel from the pre-branching vessel until substantially only the at least first flange member is still positioned within the pre-branching vessel;

(iv) withdrawing the introducer catheter to expose the at least first flange member of the intraluminal stent;

(v) causing or allowing the at least first flange member to move from its radially compressed state to its radially expanded state such that it is caused to abut with at least a portion of the wall of the pre-branching vessel which surrounds the opening of the post-branching vessel;

(vi) advancing the placement catheter and the membrane further into the post-ranching vessel such that the compression on the portion of the tubular body substantially surrounded by the membrane, by said membrane is released, allowing said portion of the tubular body to move from its radially compressed state to its radially expanded state and into abutment with at least a portion of the wall of the post-branching vessel; and

(vii) withdrawing the placement catheter together with the membrane through the expanded tubular body.

In a fifth aspect the present invention provides a delivery system for the delivery of an intraluminal stent to a target vessel, said delivery system comprising an introducer catheter having an elongate tubular body to allow the passage therethrough of a placement catheter, said placement catheter having an elongate body which extends from a proximal end to a distal end and which carries the intraluminal stent at a position intermediate said proximal end and said distal end; the delivery system further including a membrane which engages at least a portion of the intraluminal stent such that said portion of the stent is prevented from moving from a first radially compressed state to a second radially expanded state.

In this fifth aspect the stent can have the features of the stent according to the first aspect of the invention defined herein.

Preferably, the intraluminal stent is made from a shape memory material such as Nitinol™. In this embodiment, where the first flange member is present and when the introducer catheter is withdrawn to expose the at least first flange member of the intraluminal stent, said first flange member is exposed to the body temperature of the patient whereupon it moves to its “memorised” position, that is, flaring outwardly from the remainder of the tubular body of the stent. In the treatment of ostial stenosis, this forms the anchor for the stent as the flange member engages with the wall of the pre-branching vessel around the ostium of the post-ranching vessel.

To ensure that the stent is appropriately positioned before the introducer catheter is withdrawn, the at least first flange member may have radio-opaque markers incorporated in its structure. Accordingly, in this embodiment, the surgeon would be able to determine the exact positioning of the at least first flange member within a vessel(s) of the patient. Not until the at least first flange member was positioned within the pre-branching vessel at an area adjacent the opening of the post-branching vessel and the remainder of the tubular body extending into the post-branching vessel would the introducer catheter be withdrawn.

As discussed above, the remainder of the tubular body of the stent which is positioned within the post-branching vessel is typically engaged by the membrane. In this regard, the membrane may extend around the circumference of the tubular body.

The membrane is preferably made from a suitably strong material to act as a compressive force upon the tubular body thereby preventing the tubular body from moving into the radially expanded state.

The membrane may be made of a biodegradable material and could therefore be left within the body of the patient although preferably, the membrane is also withdrawn with the placement catheter. Whichever arrangement is chosen, the effect is to release the pressure exerted on the tubular body such that it can move to its radially expanded state.

In the case of use of the stent in the treatment of ostial stenosis, the membrane preferably engages with all of the tubular body of the stent apart from the at least first flange member. Accordingly, when the introducer catheter is withdrawn, the at least first flange member is free to move from its radially compressed state to its radially expanded state. In this embodiment, it is envisaged that the membrane around the at least one portion of the tubular body may be broken to enable the at least one portion of the tubular body to move from its radially compressed state to its radially expanded state.

In one embodiment, the placement catheter may include a balloon member positioned at least partially internal the tubular body of the intraluminal stent. Upon inflation of the balloon member, the intraluminal stent is forced radially outwardly which has the effect of breaking the membrane. With the membrane broken, the at least one portion of the tubular body is free to move into its radially expanded state. It is further envisaged that the membrane may include a frangible region which breaks upon the exertion of pressure caused by the inflation of the balloon member.

It is also further envisaged that the membrane may not break but rather the membrane is caused to radially expand with the radial expansion of the tubular body of the intraluminal stent. Such radial expansion may result from the force exerted by the inflation of the balloon member.

As discussed above, the membrane may be biodegradable in which case, upon placement of the tubular body in a target vessel, the membrane may degrade thereby allowing the tubular body to move to its radially expanded state.

The membrane can deliver any of a number of pharmaceutical agents to a target vessel. In this embodiment, the membrane may be coated with such agents. Furthermore, the membrane may comprise a number of layers carrying different pharmaceutical agents. Each layer may be biodegradable to expose another layer.

In a sixth aspect, the present invention provides a delivery system for the delivery of an intraluminal stent to a target vessel, said intraluminal stent being movable from a first radially compressed state to a second radially expanded state at a target site within the vessel, said delivery system comprising a catheter which in turn comprises an elongate body which extends from a proximal end to a distal end wherein the elongate body extends through an internal lumen of the intraluminal stent such that said intraluminal stent substantially surrounds a portion of the catheter; the delivery system further comprising at least one compression member which holds the intraluminal stent in its first radially compressed state and a release member which causes release of the compression member and allows the intraluminal stent to move to its second radially expanded state.

It is preferred that the delivery system is used to deliver a self expanding intraluminal stent to a target site.

In one embodiment, the compression member of the sixth aspect forms a membrane around the intraluminal stent. In this regard, the membrane is made from a suitably strong yet flexible material to compress the intraluminal stent and prevent said stent moving from its first radially compressed state to its second radially expanded state.

In a further embodiment, it is envisaged that the compression member includes a tie member or a series of tie members which hold the intraluminal stent around the catheter and prevent the intraluminal stent moving from its first compressed state to its second expanded state. For example, the tie member(s) may be sutures which have a pre-determined breaking strength. The sutures may be bonded to the intraluminal stent such that they do not form free particles during and after the deployment of said stent.

Alternatively, the compression member may comprise one or more of a collar, ring or spiral wrap or combinations thereof around the intraluminal stent.

The advantage of the delivery system of the sixth aspect is that the self expanding intraluminal stent may be delivered to a target site without the requirement of a bulky introducer catheter to hold the intraluminal stent in its first radially compressed state. In this regard, the compression member of the delivery system holds the intraluminal stent in its radially compressed state until the stent is advanced on the catheter to the target site.

When the intraluminal stent reaches the target site, the release member may be activated to release the compressive force of the compression member on the intraluminal stent. In this regard, the release member may comprise a balloon member positioned along the length of the elongate body of the catheter. The balloon member may form the portion of the elongate body of the catheter which is substantially surrounded by the intraluminal stent. The balloon member may move from a deflated state during delivery of the catheter to a target site, to an inflated state at said target site. Movement of the balloon member to the inflated state may release the compressive force of the compression member.

Particularly, where the compression member is a membrane which surrounds the intraluminal stent, it is preferred that said membrane includes at least one frangible region such that when the balloon member moves from its deflated to inflated state, the frangible region(s) is broken and the intraluminal stent allowed to move to its second radially expanded state. In this regard, in a preferred embodiment wherein the intraluminal stent is a self expanding stent, breaking the compression member at the frangible region(s) allows said stent to spring to its second radially expanded state, that is, the balloon member is typically inflated only a sufficient degree to break the engagement between compression member and stent and need not be inflated further to cause the intraluminal stent to move from its first radially compressed state to its second radially expanded state. In one embodiment, said frangible region may comprise one or more perforations, such as a line of perforations, in the body of the membrane.

In the embodiment wherein the compression member comprises a number of sutures, as mentioned above, the sutures holding the intraluminal stent to the catheter have a predetermined breaking strength. Accordingly, when the balloon member moves from its deflated state to its inflated state, the sutures break thereby allowing the intraluminal stent to move to its second radially expanded state.

Similarly, in further embodiments, the movement of the balloon member from its deflated to its inflated state may cause said one or more of the collar, ring or spiral wrap to release their compressive force.

In another embodiment, where the compression member forms a membrane around the intraluminal stent, it is envisaged that said membrane has a perforation along at least a portion of its length. The release member in this embodiment may be a pull suture which is aligned with and/or threaded through the perforation of the membrane. The pull suture typically extends to a location outside the body or is connected to an actuator member located outside the body such that when the intraluminal stent is in position at a target site, the pull suture is drawn in a direction towards the proximal end of the intraluminal stent such that the perforation is broken and the compressive force of the membrane released from the intraluminal stent which may then move to its radially expanded state.

In a further embodiment of this aspect, the compression member is an expandable membrane, The membrane can be sealed around the stent. In this aspect, the release member comprises a fluid that can be delivered to the sealed membrane and so expand the membrane.

In one embodiment, the fluid can be delivered into the sealed membrane by the catheter through apertures therein.

In a preferred embodiment, the membrane breaks into one or more portions on undergoing a predetermined degree of expansion. Said one or more portions of the membrane can be connected to the catheter and are withdrawable from the target vessel on withdrawal of the catheter therefrom.

In a further embodiment, the membrane can contain or include one or more pharmaceutical agents. In another embodiment, one or more pharmaceutical agents can be delivered with the fluid from the catheter arid into the pocket formed by the sealed membrane.

In a further embodiment, the stent can be a self-expanding stent that expands on expansion of the membrane. In another embodiment, the stent is a passive stent that requires expansion by a secondary mechanism, such as a balloon catheter.

### BRIEF DESCRIPTION OF THE DRAWINGS

FIG. 1 a is a cut-away view of anatomical structures of the abdomen of a subject.

FIG. 1b is a magnified view of part of the structure depicted in FIG. 1a.

FIG. 2 Is a perspective view of one embodiment of the intraluminal stent of the invention.

FIG. 3a is a view of the cell structure of the intraluminal stent depicted in FIG. 2 when the stent is in an expanded state.

FIG. 3b is a view of the cell structure of the intraluminal stent depicted in FIG. 2 when the stent is in a compressed state.

FIG. 4 is a perspective view of another embodiment of the intraluminal stent of the invention.

FIG. 5a is a view of the cell structure of the intraluminal stent depicted in FIG. 4 when the stent is in an expanded state.

FIG. 5b is a view of the cell structure of the intraluminal stent depicted in FIG. 4 when the stent is in a compressed state.

FIG. 6 is a perspective view of a further embodiment of the intraluminal stent of the invention.

FIG. 7a is a view of the cell structure of the intraluminal stent depicted in FIG. 6 when the stent is in an expanded state.

FIG. 7b is a view of the cell structure of the intraluminal stent depicted in FIG. 6 when the stent is in a compressed state.

FIG. 8 is a side elevational schematic view of an embodiment of the invention.

FIG. 9 is a side elevational schematic view of a further embodiment of the present invention.

FIG. 10 is a cross-sectional view of a further embodiment of the present invention.

FIG. 11 is a schematic view of a delivery catheter according to the present Invention.

FIGS. 12a to 12d depict placement of the intraluminal stent according to the present invention in a vessel using the delivery catheter shown in FIG. 11.

FIGS. 13a, 13b and 13c are cross-sectional views of further embodiments of the delivery catheter shown in FIG. 11.

FIGS. 14a, 14b and 14c are views of further embodiments of the present invention.

FIG. 14d shows the cells structure of the embodiment of the invention depicted in FIG. 14b.

FIGS. 15a and 15b show schematic views of a delivery system of further aspect of the invention.

FIG. 15c is a cross-sectional view showing the intraluminal stent and compression member of the delivery system depicted in FIG. 15b.

FIG. 16 is a schematic view of a further embodiment of the delivery system of the invention.

FIGS. 17a and 17b are schematic views of a further embodiment of a delivery system according to the present invention.

FIGS. 18a and 18b are schematic views of a still further embodiment of a delivery system according to the present invention.

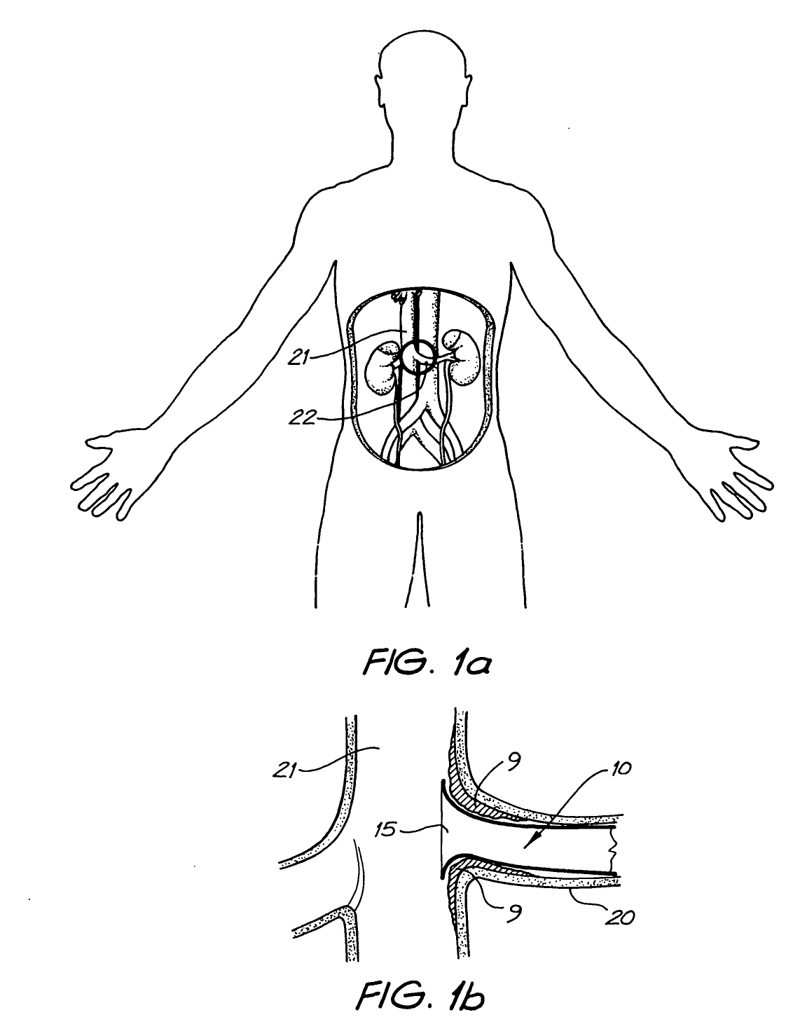

### PREFERRED MODE OF CARRYING OUT THE INVENTION

The intraluminal stent of the present invention is generally depicted as 10 in the accompanying drawings.

Preferably, the intraluminal stent of the present invention is used in the treatment of ostial stenosis although it is equally envisaged that it could be used in the treatment of any other form of stenosis. In ostial stenosis, the plaque or stenotic region 9 is formed at the junction between a pre-branching vessel 21 such as the aorta and a post-branching vessel 20 such as the renal artery which has the effect of narrowing the opening (ostium) of the post-branching vessel 20.

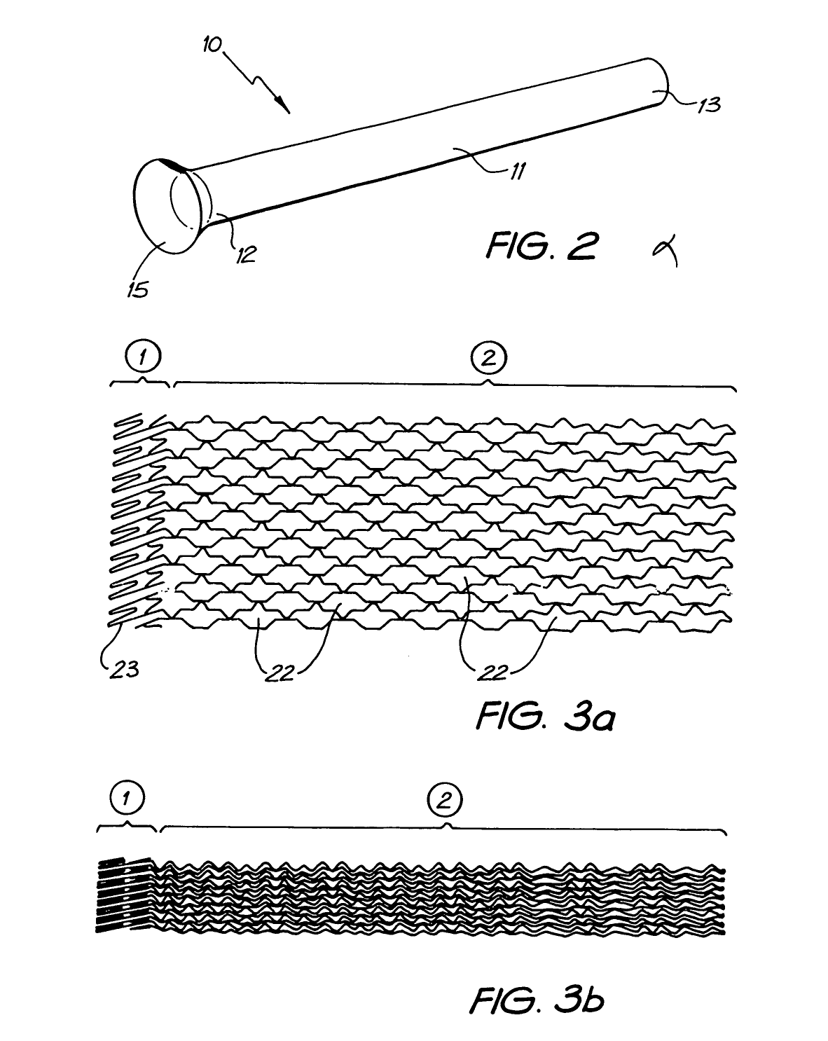

The intraluminal stent 10 comprises a tubular body 11 extending from a proximal end 12 to a distal end 13. The tubular body 11 is capable of expanding or being expanded from a radially compressed state (as depicted In FIGS. 3b, 5b and 7b) to a radially expanded state (as depicted in FIGS. 3a, 5a and 7a). When in the radially expanded state, the tubular body 11 includes a first flange member 15 positioned adjacent the proximal end 12 of the tubular body 11, with the first flange member 15 having a greater diameter than the diameter of the remainder of the tubular body 11.

The first flange member 15 extends outwardly and away from the proximal end 12 of the tubular body 11 giving the stent a trumpet-like shape as depicted in FIG. 2.

The first flange member 15 is shown as forming a continuous, integral structure with the tubular body 11 and is typically made from the same material as the remainder of the tubular body 11.

During use of the intraluminal stent 10 (in the present case, the term “use” refers to the treatment of ostial stenosis), the tubular body 11 is initially in the radially compressed state to enable delivery of the stent 10 through an introducer catheter. Upon deployment of the stent 10 into a selected vessel, the tubular body may be caused to expand, or may be allowed to self-expand into the expanded state. The first flange member 15 is positioned at least partially within pre-branching vessel 21 and the remainder of the tubular body of the stent extends into the post-branching vessel 20.

In use, the first flange member 15 is positioned in the pre-branching vessel 21, such as, for example, the aorta. The first flange member 15 typically engages at least a portion of the pre-branching vessel wall surrounding the ostium of the post-branching vessel 20 with the remainder of the tubular body 11 extending Into the post-branching vessel 20. Accordingly, the first flange member 15 has the effect of anchoring the stent 10 within the post-branching vessel 20 thereby preventing longitudinal movement of the stent into the post-branching vessel 20.

While the tubular body 11 of the stent may be formed of a thin biocompatible material such as Nitinol™ or stainless steel, other alloys such as tantalum or Elgiloy may be used. In the examples depicted, the stent is made from Nitinol™.

As depicted in FIGS. 3a, 3b, 5a, 5b and 7a, 7b, a cylinder of Nitinol™ is laser cut to form a series of cells 22. The cylinder is then taken to the desired temperature to allow the material to achieve a “memory” of a certain configuration. At this temperature, a template is used to push the area of the cylinder 23 which is to become the first flange member 15 outwardly from the remainder of the cylinder. The material of the cylinder is then cooled from this temperature such that the material resumes its cylindrical shape without the flange member. When the final stent 10 made from this cylinder is inserted into a vessel of a patient, the material takes on its “memorised” shape, that is, a tubular body having a first flange member positioned at least adjacent the proximal end 12 of the tubular body 11.

In an embodiment wherein the material of the stent is not a shape memory material, it is envisaged that a pre-formed tubular body having at least a first flange member positioned at least adjacent the proximal end of the tubular body is formed. The pre-formed tubular body may then be laser cut into a desired series of cells.

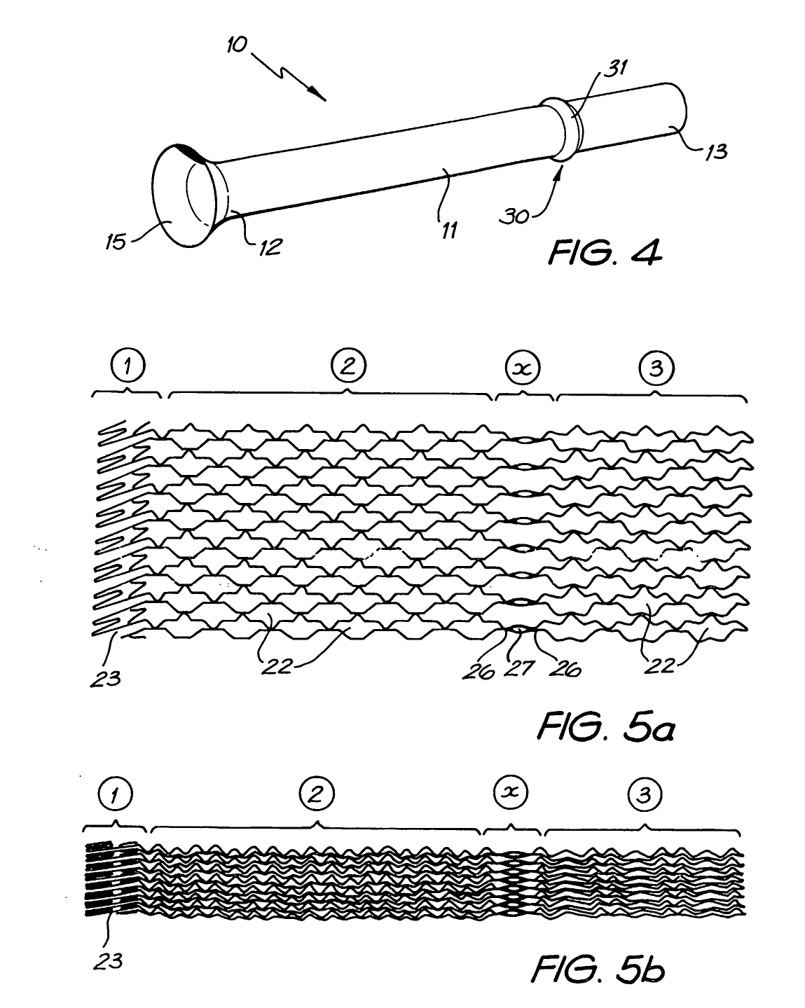

As depicted in the figures, the cells 22 of the tubular body 11 may be of varying size or configurations along the length of the tubular body 11. In each of the figures, the first flange member 15 is made up of a series of elongate cells 23 which are angled from the remainder of the cells of the tubular body. This region is generally depicted as (1) in the drawings. The region adjacent the first flange member 15 is depicted as (2). As can be seen, the cells of this region are generally of a large “bat-wing” shape which confer the desired flexibility while at the same time a certain degree of rigidity to the stent 10. In FIG. 3a, region (2) extends from a region adjacent the first flange member 15 along the remainder of the length of the tubular body 11. In FIG. 5a, region (2) is interrupted by region (x) which comprises a ring of cells 25 around the tubular body 11, wherein each cell is formed from a straight link 26 with a central hole 27. In one embodiment, wherein the material of the stent is not a necessarily a shape memory material, when the stent 10 is moved from a compressed state to an expanded state, the cells on either side of region (x) are forced closer together thereby causing the straight links 26 to buckle outwardly of the tubular body 11 at a fold adjacent the central hole 27. This creates a ridge 31 on the tubular body 11 which is depicted in FIG. 4 and will be discussed in further detail below.

Where the tubular body 11 is made from a shape memory material such as Nitinol™, a cylinder of Nitinol™ is taken to a desired temperature to allow the material to achieve a “memory” of a certain configuration. If a ridge 31 as depicted in FIG. 4 is to be formed, when the cylinder is taken to this temperature, a template is used to push the area of the cylinder which is to become the ridge 31 outwardly from the remainder of the cylindrical tubular body. The material of the cylinder is then cooled from this temperature such that the material resumes its cylindrical shape without the ridge 31. When the final stent 10 made from this cylinder is inserted into a vessel of a patient, it is preferable that when the material is exposed to the body temperature of the patient it takes on its “memorised” shape, that is, a tubular body 11 having a ridge 31 as depicted in FIG. 4.

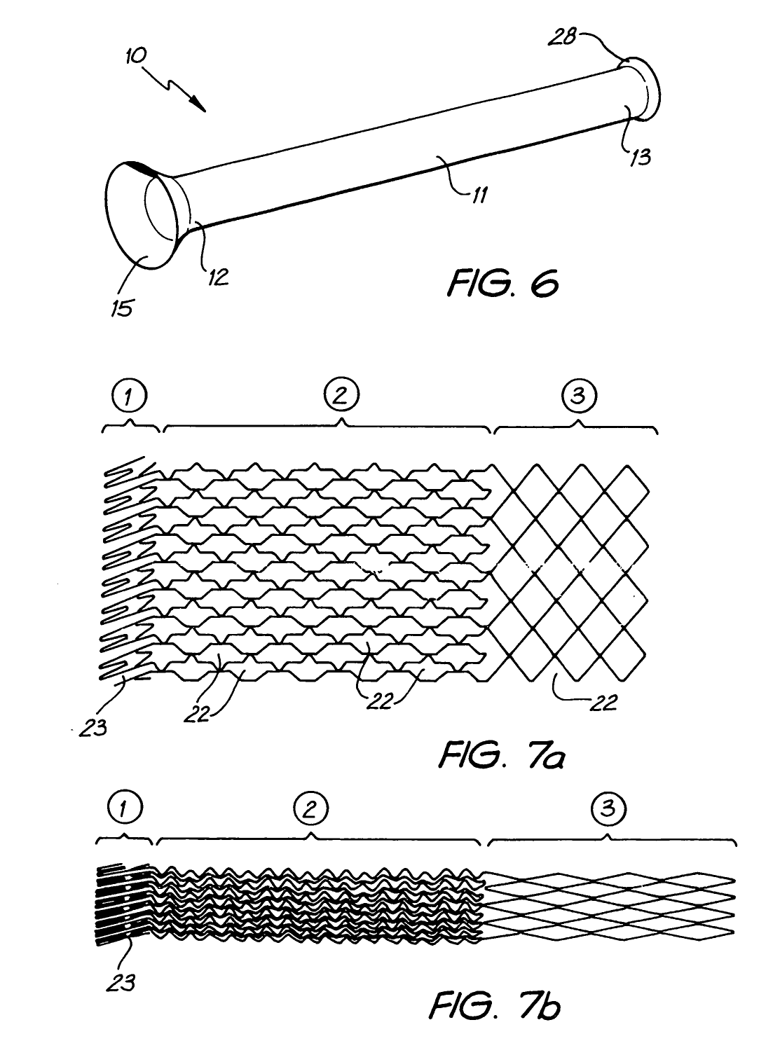

In a further embodiment of the invention as depicted in FIGS. 6, 7a and 7b, the stent 10 has a first flange member 15 and a second flange member 28. In this embodiment, the cells 23 of the first flange member 15 are of an elongate shape and make up region (1). Region (2) of this embodiment comprises a series of “bat-wing” cells 22 which extend into from region (1) to region (3). Region (3) comprises the second flange member 28 and is made of a series of relatively large diagonal-shaped cells. Where the tubular body 11 is made from a shape memory material such as Nitinol™, the second flange member 28 may be formed In the same manner as described above for the first flange member 15.

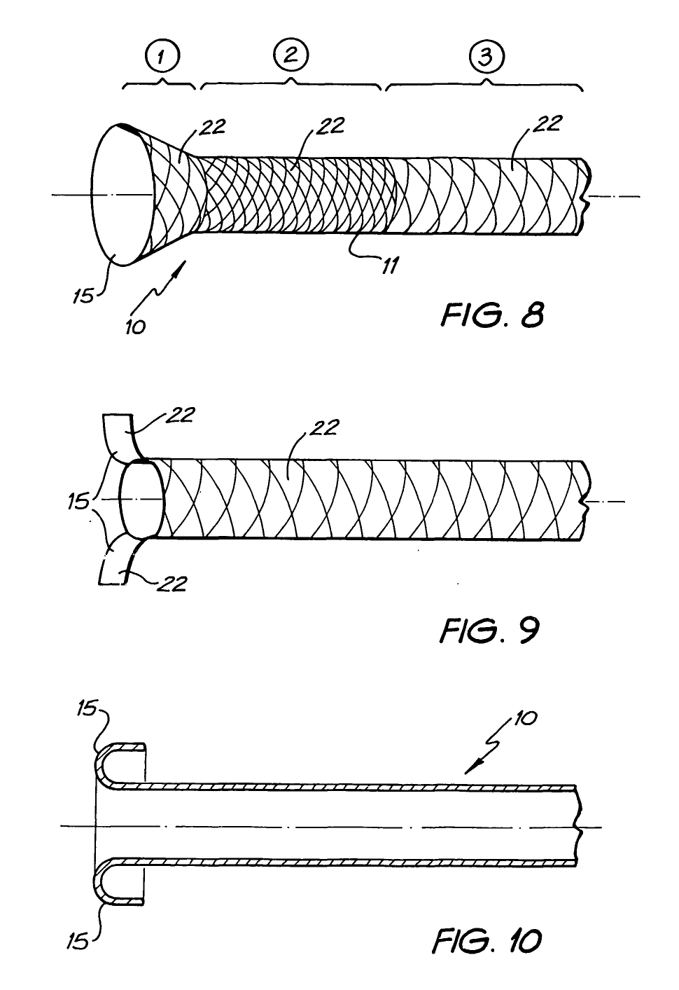

In another embodiment of the invention as depicted in FIG. 8, the tubular body is made up of regions (1), (2) and (3) wherein region (1) comprises the first flange member 15, Region (2) is made up of a series of cells 22 having a smaller, tighter pore size than the cells 22 of regions (1) and (3). This provides a region of relatively high expansile strength which acts to exert a force on a surrounding stenotic region when the stent is in use.

In one embodiment as depicted In FIG. 9, the first flange member 15 comprises two cells 22 located on opposing walls of the proximal end 12 of the tubular body 11. In this embodiment, the two cells 22 form struts to engage the area surrounding the ostium of a post-branching vessel 20.

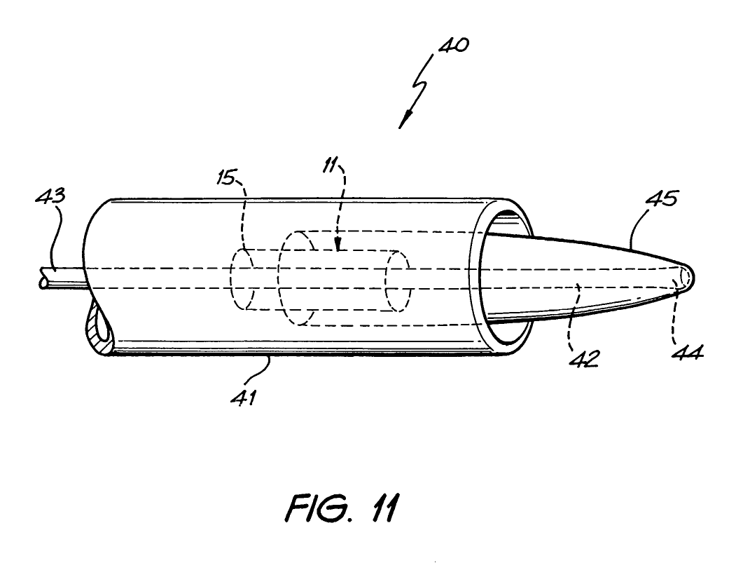

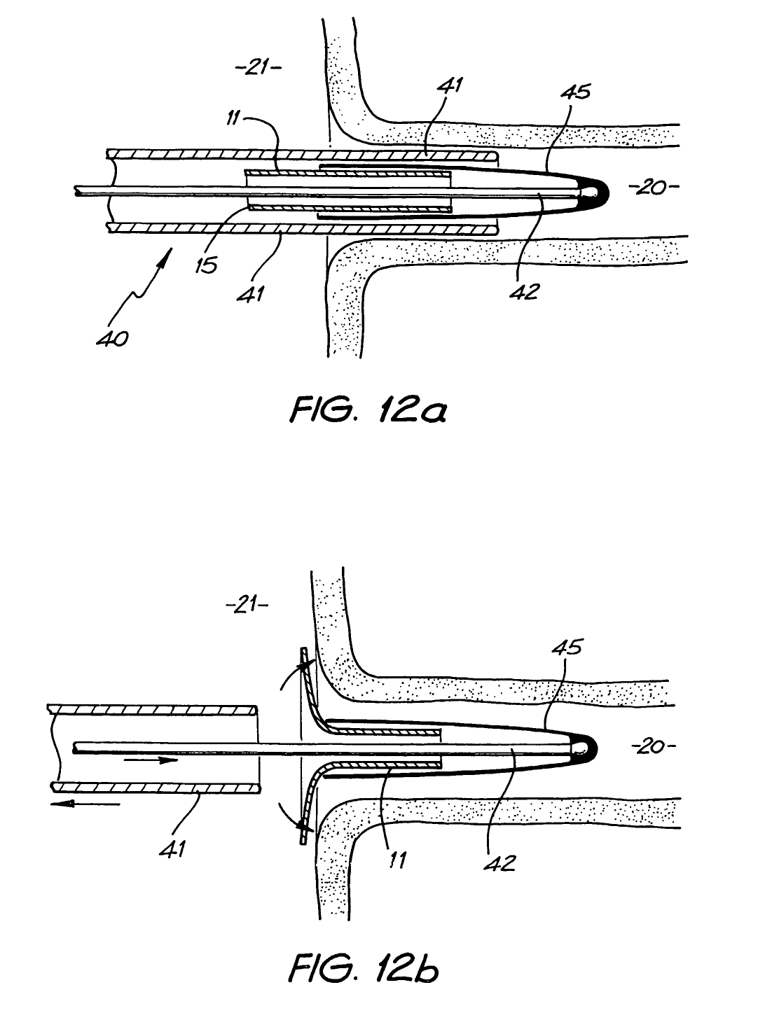

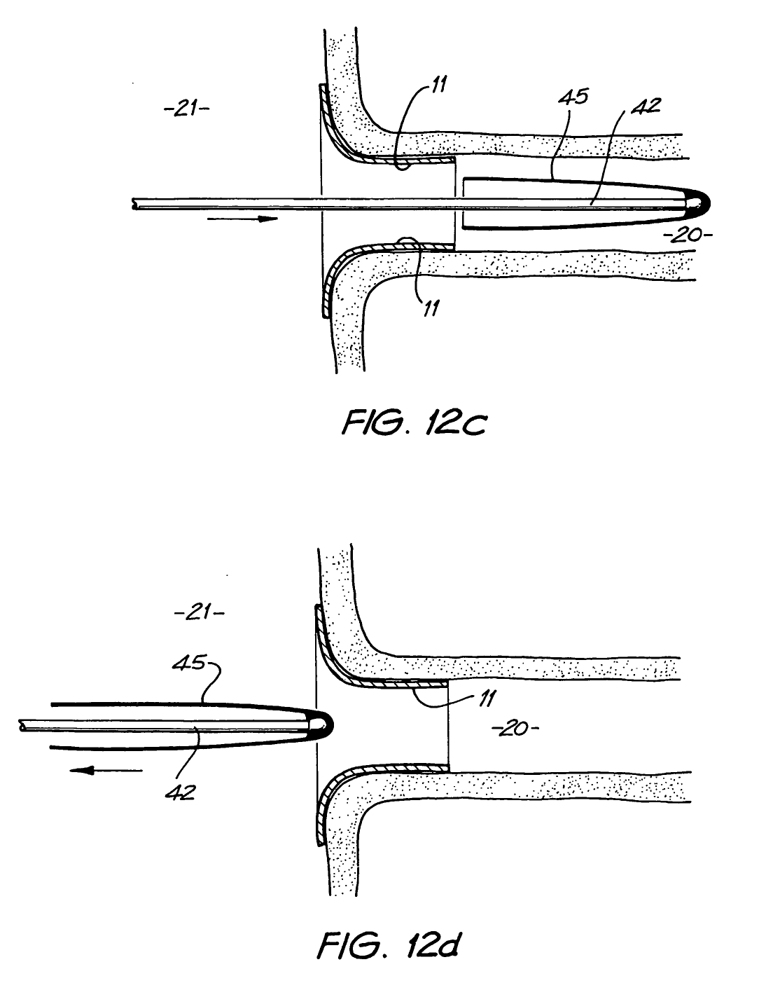

In a particularly preferred embodiment of the invention as depicted in FIG. 10, the first flange member 15 in addition to extending outwardly and away from the proximal end 12 of the tubular body 11, doubles back on itself in a direction generally towards the distal end 13 of the tubular body 11. The first flange member 15 in this embodiment therefore forms a lipped structure which provides a good anchorage of the stent 10 when it is in use. Particularly, the lipped portion engages a region of the pre-branching vessel 21 around the ostium of the post branching vessel 20 thereby preventing movement of the stent 10 into the post-branching vessel 20 such that the stent does not bridge an area of stenosis around the ostium of the post-branching vessel.

As depicted in FIG. 4, the tubular body 11 also includes an engagement member 30. The engagement member 30 is connected to or integral with a wall of the tubular body 11 at a position located intermediate the proximal end 12 and the distal end 13 of the tubular body 11.

The engagement member 30 depicted in FIG. 4 is ridge 31. When the stent 10 is in use, the ridge 31 extends towards and engages with the post-branching vessel 20 wall thereby securing the stent in the post-branching vessel 20.

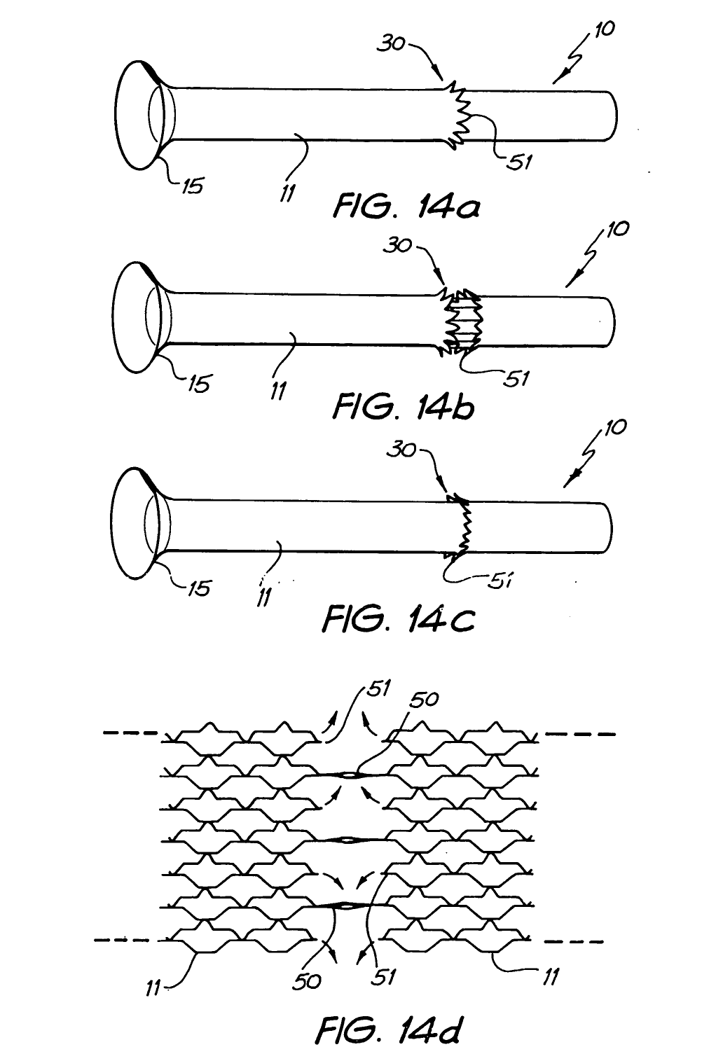

In a further embodiment depicted in FIGS. 14a-14d, the engagement member 30 may be made up of a series of connector members 51 which connect the cells on either side of the at least one engagement member 30. In this regard, the connector members 51 may be relatively straight members and may connect every second cell on either side of the at least one engagement member. In this embodiment, when the tubular body moves from the radially compressed state to the radially expanded state, the free ends 51 of every second cell which are not connected by the connector members 50 turn outwardly away from the tubular body and are engageable with the vessel wall.

As mentioned above, the stent 10 of the present invention is used in the treatment of ostial stenosis including ostial stenosis of the visceral arteries such as the renal and mesenteric arteries, the iliac artery and the sub-clavian artery. Further, in addition to the treatment of stenotic lesions in the peripheral vasculature, the stent 10 may be used in the treatment of vessels comprising the coronary circulation.

FIG. 11 shows a delivery catheter 40 for the delivery of the intraluminal stent 10. The delivery catheter 40 includes an introducer catheter 41 having an elongate tubular body to allow the passage therethrough of a placement catheter 42. The placement catheter 42 has an elongate body extending from a proximal end 43 to a distal end 44, the placement catheter carrying the stent 10 having body 11 at a position intermediate its proximal end 43 and its distal end 44. The delivery catheter 40 further includes a membrane 45 positioned around the placement catheter 42 and connected to distal end 44 wherein the membrane 45 is positioned such that it does not surround the first flange member 15 of the intraluminal stent 10 and wherein the membrane 45 acts to compress the remainder of the tubular body and prevent it from moving to the radially expanded state.

When the intraluminal stent 10 is to be used to bridge an ostial stenosis, it is introduced as follows:

Step 1: the introducer catheter 41 is introduced by way of a vein, artery or other vessel into the pre-branching vessel 21 of a patent. It should be noted that the tubular body of the intraluminal stent is in its radially compressed state;

Step 2: the distal end 44 of the placement catheter 42 is introduced into the post-branching vessel 20 from the pre-branching vessel 21 until substantially only the first flange member 15 is still positioned within the pre-branching vessel 21;

Step 3: the introducer catheter 41 is pulled back to expose the first flange member 15 of the intraluminal stent 10;

Step 4: the first flange member 15 moves from its radially compressed state to its radially expanded state such that it abuts with at least a portion of the wall of the pre-branching vessel 21 which surrounds the ostium of the post-branching vessel 20. It should be noted that in the example depicted, the stent is made from Nitinol™. Accordingly, in its first radially compressed state, the first flange member 15 simply forms part of the cylinder (as shown in FIG. 11). Being exposed to the surrounding body temperature, the flange member 15 takes on its “memorised” expanded state which is the outwardly flared structure described above. The expansion of the first flange member 15 anchors the stent 10 such that the remainder of the tubular body 11 cannot move any further downstream within the post-branching vessel 20;

Step 5: the placement catheter 42 and the membrane 45 are advanced downstream into the post-branching vessel 20 such that the portion of tubular body previously compressed by the membrane 45 is caused or allowed to move from its radially compressed state to its radially expanded state such that it is caused to abut with at least a portion of the wall of the post-branching vessel 20;

Step 6: withdrawing the placement catheter 42 together with the membrane 45 through the expanded tubular body 11. In this regard, it is preferred that the membrane 45 is spring connected to the placement catheter such that it is biased against the placement catheter 42. This has the advantage that the membrane 45 is held against the placement catheter 42 during withdrawal of the placement catheter 42 thereby preventing snagging of the membrane on the radially expanded stent 10.

To ensure that the stent 10 is appropriately positioned before the introducer catheter 41 is withdrawn, the first flange member 15 may have radio-opaque markers Incorporated in its structure. Accordingly, the surgeon, would be able to determine the exact positioning of the first flange member 15 within the vessel of the patient. Not until the at least first flange member 15 is positioned within the pre-branching vessel 21 at an area adjacent the opening of the post-branching vessel 20 would the introducer catheter 41 be withdrawn.

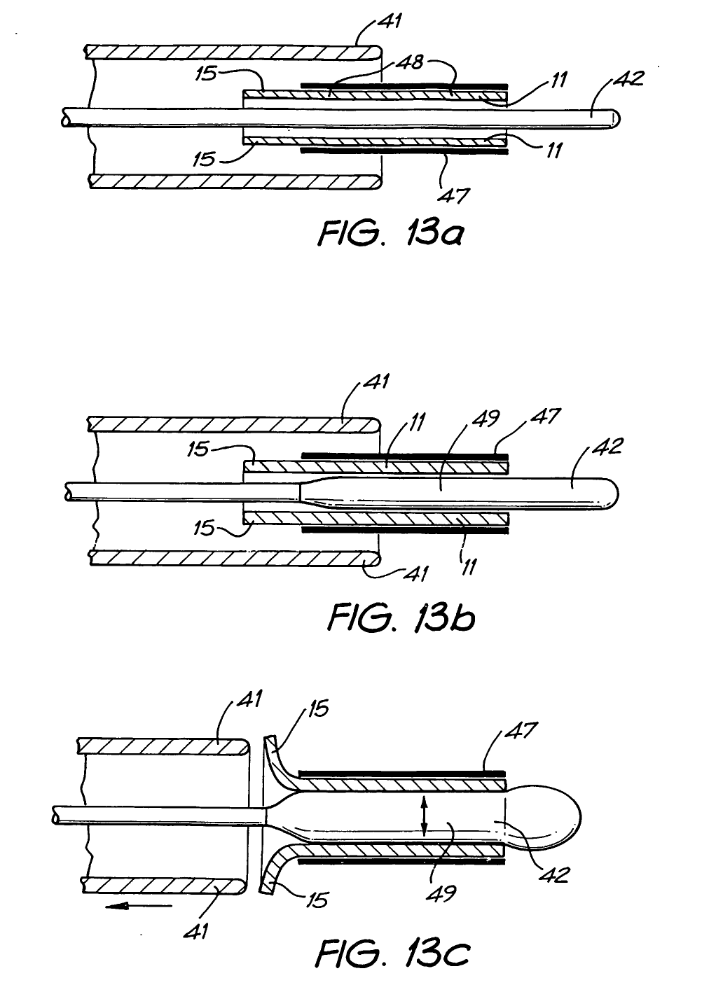

In a further embodiment of the invention as depicted in FIG. 13a, the system has a membrane 47 which engages with at least a portion 48 of the intraluminal stent 10 and maintains said portion in its radially compressed state. As depicted, the membrane 47 engages with all of the tubular body 11 of the stent 10 apart from the first flange member 15. Accordingly, when the introducer catheter 41 is withdrawn, the first flange member 15 is free to move from its radially compressed state to its radially expanded state.

In the embodiment depicted in FIGS. 13b and 13c, the placement catheter 42 includes a balloon member 49 positioned at least partially internal the tubular body 11 of the intraluminal stent 10. Upon inflation of the balloon member 49, the tubular body 11, is forced radially outwardly which has the effect of breaking the membrane 47. With the membrane 47 broken, the at least one portion 48 of the tubular body 11 is free to move into its radially expanded state.

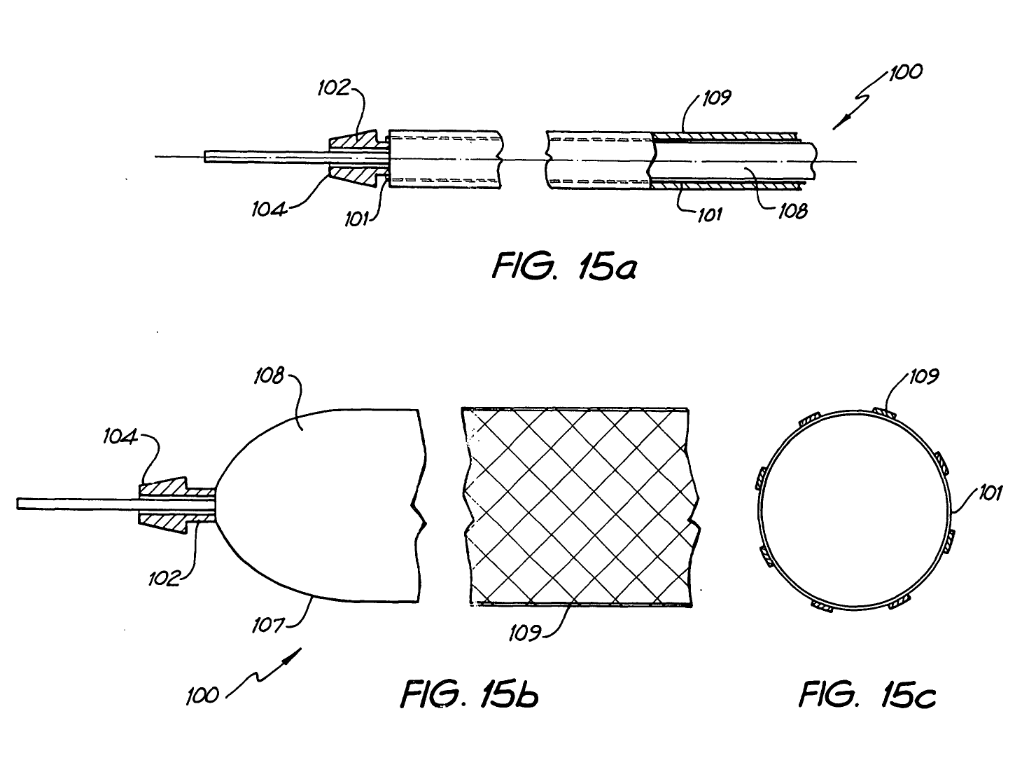

The delivery system of a further aspect of the invention is generally depicted as 100 in FIGS. 15-17. The delivery system 100 is used for the delivery of an intraluminal stent 101 to a target vessel.

As depicted, the intraluminal stent 101 is movable from a first radially compressed state (see FIG. 15a) to a second radially expanded state (see FIG. 15b).

The delivery system 100 includes a catheter 102 comprising an elongate body which extends from a proximal end 104 to a distal end 105. The elongate body extends through an internal lumen of the intraluminal stent 101 such that the intraluminal stent 101 substantially surrounds a portion of the catheter 102.

The delivery system 100 further includes a compression member which holds the intraluminal stent 101 in its first radially compressed state and a release member 107 which causes release of the compression member and allows the intraluminal stent to move to its second radially expanded state.

The delivery system 100 is used to deliver a self-expanding intraluminal stent to a target site.

As shown in FIGS. 15a-15c, the compression member is a membrane 109 around the intraluminal stent. In this regard, the membrane 109 is made from a suitably strong yet flexible material to compress the intraluminal stent and prevent the stent moving from its first radially compressed state to its second radially expanded state.

The release member 107 comprises a balloon member 108 positioned along the length of the elongate body of the catheter 102. The balloon member 108 is substantially surrounded by the intraluminal stent 101 and when the balloon member 108 is inflated the intraluminal stent is forced radially outward.

The membrane 109 includes a series of perforations along its. length which are broken upon inflation of the balloon member 108 (as shown in FIG. 15c). FIG. 15c depicts the expanded intraluminal stent 101 and the separated portions of the membrane 109. As shown, the separated portions of the membrane 109 remain attached to the stent 101 on expansion of the stent. In another embodiment, the membrane 109 is attached at its proximal end to the catheter 102 such that it may be withdrawn from the target vessel with the catheter 102.

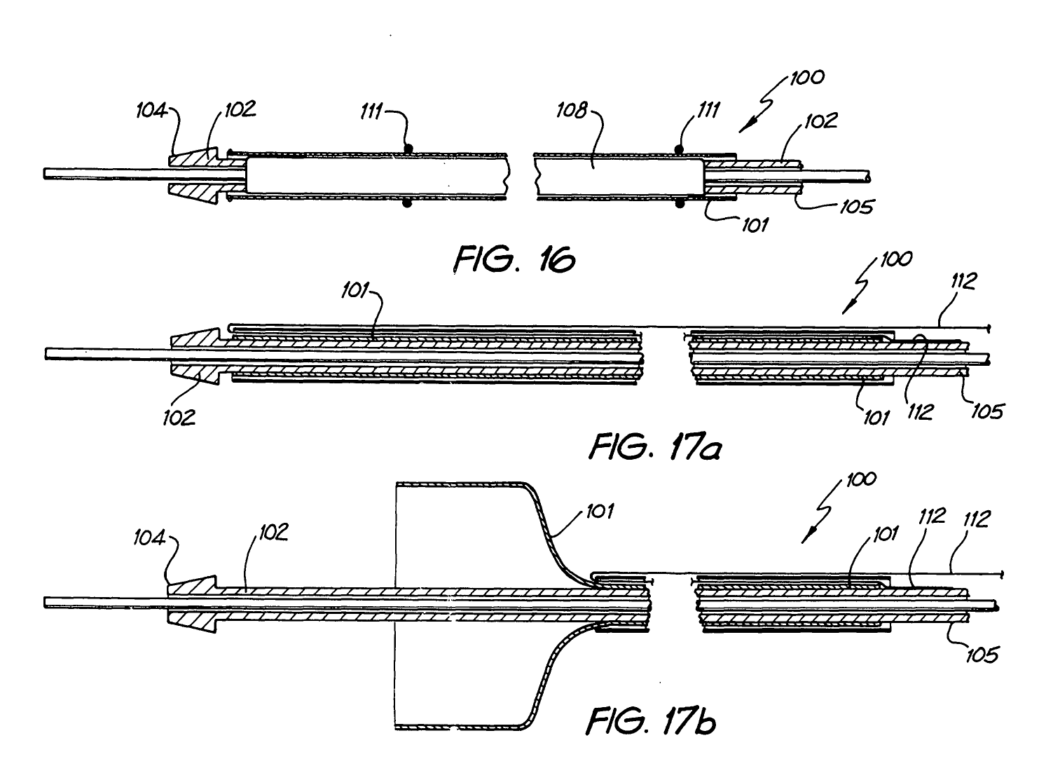

The delivery system of FIG. 16 operates in a manner similar to that described above except that the compression member comprises a number of tie members or sutures 111 which hold the intraluminal stent 101 around the catheter 102 and prevent the intraluminal stent 101 moving from its first compressed state to its second expanded state.

In this embodiment, the sutures 111 holding the intraluminal stent 101 to the catheter 102 have a predetermined breaking strength. Accordingly, when the balloon member 108 moves from its deflated state to its inflated state, the sutures 111 break thereby allowing the intraluminal stent 101 to move to its second radially expanded state.

Another embodiment of a delivery system is depicted in FIGS. 17a to 17b. In this embodiment the compression member comprises a membrane 109 around the intraluminal stent 101. The membrane has a perforation along at least a portion of its length. The release member in this embodiment is a pull suture 112 which is aligned with and/or threaded through the perforation of the membrane 109. The pull suture 112 typically extends to a location outside the body or is connected to an actuator member located outside the body such that when the intraluminal stent 101 is in position at a target site, the pull suture 112 is drawn in a direction towards the proximal end of the intraluminal stent 101 such that the perforation is broken and the compressive force of the membrane 109 released from the intraluminal stent 101 which may then move to its radially expanded state.

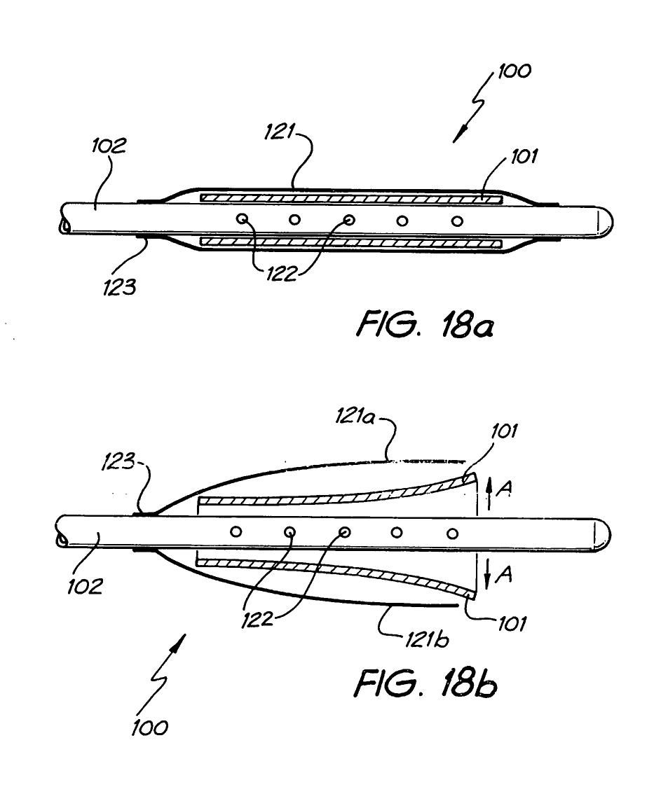

In the embodiment of the delivery system depicted in FIG. 18, the compression member is an expandable membrane 121. The membrane 121 is sealed around the stent 101 on delivery of the stent 101 into the target vessel.

The release member comprises a fluid that can be delivered to the sealed membrane 121 and so expand the membrane 121. The fluid is delivered into the sealed membrane 121 by the catheter 102 through apertures 122 therein.

The membrane 121 breaks into one or more portions 121a and 121b on undergoing a predetermined degree of expansion. In the depicted embodiment, the proximal end 123 of the portions are connected to the catheter 102 and are withdrawable from the target vessel on withdrawal of the catheter 102 therefrom.

In this embodiment, the membrane 121 can contain or include one or more pharmaceutical agents. In another embodiment, one or more pharmaceutical agents can be delivered with the fluid from the catheter 102 and into the pocket formed by the sealed membrane 121.

In the depicted embodiment, the stent 101 is a self-expanding stent that expands in the direction depicted by arrows A on expansion of the membrane 121. It will be appreciated that in another embodiment, the stent can be a passive stent that requires expansion by a secondary mechanism, such as a balloon catheter.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1-54. (canceled).

55. An intraluminal stent comprising a tubular body extending from a proximal end to a distal end, said tubular body being capable of expanding or being expanded from a radially compressed state to a radially expanded state wherein, when at least in the radially expanded state, the tubular body comprises at least a first flange member positioned at or adjacent to the proximal end of the tubular body and extending outwardly from said tubular body.

56. The intraluminal stent of claim 55 wherein said first flange member comprises a main body that extends from one end that is connected to or integral with the proximal end of the tubular body to a free end of the flange member.

57. The intraluminal stent of claim 56 wherein the cross-sectional diameter of the flange member increases as it extends from said one end to said free end.

58. The intraluminal stent of claim 56 wherein the main body of the flange member extends from the tubular body at substantially right angles to said tubular body.

59. The intraluminal stent of claim 56 wherein the main body of the flange member comprises a curved member that initially extends in a linear fashion away from the proximal end of the tubular body before curling back in a direction substantially towards the distal end of the tubular body.

60. The intraluminal stent of claim 55 further comprising a second flange member positioned at or adjacent the distal end of the tubular body.

61. The intraluminal stent of claim 60 wherein said second flange member comprises a main body that extends from one end that is connected to or integral with the distal end of the tubular body to a free end of said second flange member.

62. The intraluminal stent of claim 55 when made from a shape memory material including Nitinol™.

63. The intraluminal stent of claim 55 wherein the tubular body of the stent is made up of a series of cells.

64. The intraluminal stent of claim 63 wherein the cells vary in size and configuration along the length of the tubular body.

65. The intraluminal stent of claim 64 wherein the first flange member is made from a series of cells which are larger and/or more elongate than the remainder of the cells of the tubular body.

66. The intraluminal stent of claim 65 wherein the cells of the flange member are at an angle relative to the remainder of the cells of the tubular body.

67. The intraluminal stent of claim 55 when used in the treatment of ostial stenosis including ostial stenosis of the renal and mesenteric arteries, the iliac artery, the sub-clavian artery and the coronary circulation.

68. The intraluminal stent of claim 67 wherein the first flange member is positionable within a pre-branching vessel of the ostial stenotic region and wherein said first flange member substantially engages at least a portion of the wall of the pre-branching vessel which surrounds the ostium of a post-branching vessel.

69. The intraluminal stent of claim 68 wherein the part of the tubular body of the stent not comprising the first flange member is extendable into the post-branching vessel and wherein the first flange member anchors said part of the tubular body within the post-branching vessel thereby preventing longitudinal movement of the intraluminal stent into the post-branching vessel.

70. The intraluminal stent of claim 55 wherein the tubular body of the stent further includes at least one engagement member which is connected to or integral with a wall of the tubular body at a position located intermediate the proximal end and the distal end of the tubular body.

71. The intraluminal stent of claim 55 wherein the tubular body is coated with materials to promote adhesion of cells or cell growth.

72. An intraluminal stent comprising a tubular body and a flange member connected to or integral with said tubular body, wherein the tubular body and the flange member are moveable between a radially compressed state and a radially expanded state and wherein further, when in their radially compressed states, the tubular body and the flange member comprise a stent having a substantially uniform cross-sectional diameter and when in their radially expanded states, at least a portion of the flange member has a greater cross-sectional diameter than the cross-sectional diameter of the tubular body.

73. A method of positioning the intraluminal stent of claim 55 in a vessel of a patient, the method comprising the steps of:
(i) introducing a catheter or other delivery device into a vein, artery or other vessel in the body of a patient when the tubular body of the intraluminal stent is in the radially compressed state;
(ii) causing the intraluminal stent to be carried through the catheter or other delivery device to a target site of stenosis at a bifurcation between a first pre-branching vessel and a second post-branching vessel;
(iii) causing or allowing the tubular body of the intraluminal stent to expand such that the at least first flange member is positioned at least partially within the pre-branching vessel and the remainder of the tubular body of the stent extends into the post-branching vessel; and
(iv) withdrawing the catheter or other delivery device along with any other apparatus used to introduce the intraluminal device into the vessel from the body of the patient.

74. A delivery system for the delivery of the intraluminal stent of claim 55 to a target vessel, said delivery system comprising an introducer catheter having an elongate tubular body to allow the passage therethrough of a placement catheter, said placement catheter having an elongate body which extends from a proximal end to a distal end and which carries the stent of claim 1 at a position intermediate said proximal end and said distal end, the delivery system further comprising a membrane which engages a portion of the tubular body of the intraluminal stent not comprising the at least first flange member wherein said membrane maintains said portion of the tubular body in its radially compressed state.

75. A method of delivering the intraluminal stent of claim 55 using the delivery system of claim 20, said method comprising the steps of:
(i) introducing the introducer catheter into a vein, artery or other vessel in the body of a patient wherein the tubular body of the intraluminal stent is in the radially compressed state;
(ii) causing the intraluminal stent, the placement catheter and the membrane to be carried through the introducer catheter to a target site of stenosis at a bifurcation between a first pre-branching vessel and a second post-branching vessel;
(iii) introducing the distal end of the placement catheter into the post-branching vessel from the pre-branching vessel until substantially only the at least first flange member is still positioned within the pre-branching vessel;
(iv) withdrawing the introducer catheter to expose the at least first flange member of the intraluminal stent;
(v) causing or allowing the at least first flange member to move from its radially compressed state to its radially expanded state such that it is caused to abut with at least a portion of the wall of the pre-branching vessel which surrounds the opening of the post-branching vessel;
(vi) advancing the placement catheter and the membrane further into the post-branching vessel such that the compression on the portion of the tubular body substantially surrounded by the membrane, by said membrane is released, allowing said portion of the tubular body to move from its radially compressed state to its radially expanded state and into abutment with at least a portion of the wall of the post-branching vessel; and
(vii) withdrawing the placement catheter with or without the membrane through the expanded tubular body.

76. A delivery system for the delivery of an intraluminal stent to a target vessel, said delivery system comprising an introducer catheter having an elongate tubular body to allow the passage therethrough of a placement catheter, said placement catheter having an elongate body which extends from a proximal end to a distal end and which carries the intraluminal stent at a position intermediate said proximal end and said distal end; the delivery system further comprising a membrane which engages at least a portion of the intraluminal stent such that said portion of the stent is prevented from moving from a first radially compressed state to a second radially expanded state.

77. The delivery system of claim 74 wherein the membrane extends around the circumference of the tubular body of the intraluminal stent.

78. The delivery system of claim 77 wherein the membrane is made from a suitably strong material to act as a compressive force upon the stent thereby preventing the stent from moving to its radially expanded state.

79. The delivery system of claim 74 wherein the placement catheter includes a balloon member which is positioned at least partially within an internal lumen of the tubular body of the intraluminal stent.

80. The delivery system of claim 79 wherein inflation of the balloon member forces the intraluminal stent radially outwardly thereby breaking a region of the membrane.

81. The delivery system of claim 74 wherein the membrane contains or includes a pharmaceutical agent.

82. The delivery system of claim 81 wherein said membrane comprises a reservoir member to hold the pharmaceutical agent within the membrane.

83. A delivery system for the delivery of an intraluminal stent to a target vessel, said intraluminal stent being movable from a first radially compressed state to a second radially expanded state at a target site within the vessel, said delivery system comprising a catheter which in turn comprises an elongate body which extends from a proximal end to a distal end wherein the elongate body extends through an internal lumen of the intraluminal stent such that said intraluminal stent substantially surrounds a portion of the catheter; the delivery system further comprising at least one compression member which holds the intraluminal stent in its first radially compressed state and a release member which causes release of the compression member and allows the intraluminal stent to move to its second radially expanded state.

84. The delivery system of claim 83 when used to deliver a self expanding intraluminal stent to a target site.

85. The delivery system of claim 83 wherein the compression member comprises a membrane around the intraluminal stent.

86. The delivery system of claim 83 wherein the compression member includes a tie member or a series of tie members which anchor the intraluminal stent to the catheter and prevent the intraluminal stent moving from its first compressed state to its second expanded state.

87. The delivery system of claim 86 wherein the tie member(s) are sutures which have a pre-determined breaking strength.

88. The delivery system of claim 83 wherein the compression member comprises one or more of a collar, ring or spiral wrap or combinations thereof around the intraluminal stent.

89. The delivery system of claim 83 wherein the release member comprises a balloon member positioned along the length of the elongate body of the catheter.

90. The delivery system of claim 89 wherein the balloon member is substantially surrounded by the intraluminal stent.

91. The delivery system of claim 89 wherein movement of the balloon member from a first deflated state to a second inflated state releases the compressive force of the compression member.

92. The delivery system of claim 91 wherein said membrane has at least one frangible region such that when the balloon member moves from its deflated to inflated state, said at least one frangible region is broken and the intraluminal stent is allowed to move to its second radially expanded state.

93. The delivery system of claim 92 wherein said frangible region comprises one or more perforations in the body of the membrane.

94. The delivery system of claim 83 wherein the intraluminal stent is a self expanding stent.

95. The delivery system of claim 83 wherein the compression member is a membrane around the intraluminal stent, said membrane having at least a frangible region along at least a portion of its length, and said release member is a pull suture which is aligned with and/or threaded through the frangible region of the membrane.

96. The delivery system of claim 95 wherein the pull suture extends to a location outside the body such that when the intraluminal stent is in position at a target site, the pull suture is movable in a direction towards the proximal end of the intraluminal stent such that the frangible region is broken and the compressive force of the membrane released from the intraluminal stent which may then move to its radially expanded state.

97. The delivery system of claim 83 wherein the compression member is an expandable membrane.

98. The delivery system of claim 97 wherein the membrane is sealed around the stent.

99. The delivery system of claim 98 wherein the release member comprises a fluid that can be delivered to the sealed membrane and so expand the membrane.

100. The delivery system of claim 99 wherein the fluid is delivered into the sealed membrane by the catheter through apertures therein.

101. The delivery system of claim 100 wherein the membrane breaks into one or more portions on undergoing a predetermined degree of expansion.

102. The delivery system of claim 98 wherein the membrane contains or includes one or more pharmaceutical agents.

103. The delivery system of claim 102 wherein the membrane comprises a reservoir member to hold one or more pharmaceutical agents therewithin.

104. A delivery system for the delivery of an intraluminal stent to a target vessel, said delivery system comprising an introducer catheter having an elongate tubular body to allow the passage therethrough of a placement catheter, said placement catheter having an elongate body which extends from a proximal end to a distal end and which carries the intraluminal stent at a position intermediate said proximal end and said distal end; the delivery system further including a membrane around the intraluminal stent said membrane holding the intraluminal stent in a radially compressed configuration and wherein said membrane comprises a first section and a second section, said first and second sections demarcated by a region of weakness in the membrane.
